(19) United States
(12) Patent Application Publication
(10) Pub. No.: US 2005/0085446 A1
(43) Pub. Date: Apr. 21, 2005

(54) Fluoroquinolone formulations and methods of making and using the same

(76) Inventors: M.K. Manoj Babu, Winterville, NC (US); R. Peter Thompson, Greenville, NC (US); Tapan Niranjan Godiwala, Charlotte, NC (US); Michael Ray Mascarenas, Greenville, NC (US)

Correspondence Address: MYERS BIGEL SIBLEY & SAJOVEC, PO BOX 37428, RALEIGH, NC, 27627, US

(21) Appl. No.: 10/817,507

(22) Filed: Apr. 2, 2004

Related U.S. Application Data
(63) Continuation-in-part of application No. 10/413,045, filed on Apr. 14, 2003.

(51) Int. Cl. A61K 31/724; A61K 31/573; A61K 31/60; A61K 31/496; A61K 31/4709

(52) U.S. Cl. 514/58; 514/171; 514/165; 514/253.08; 514/312; 514/570

(57) ABSTRACT

A pharmaceutical composition comprising a fluoroquinolone such as ciprofloxacin, cyclodextrin, and a hydroxy acid is described. The composition may be an aqueous composition, with such aqueous compositions preferably having a pH between 5 and 7. In some preferred embodiments, the composition further comprises a soluble polymer.

### RELATED APPLICATIONS

This application is a continuation-in-part of commonly owned, copending application Ser. No. 10/413,045, filed Apr. 14, 2003, the disclosure of which is incorporated by reference herein in its entirety.

### FIELD OF INVENTION

The present invention relates to liquid formulations, in particular pharmaceutical formulations, containing fluoroquinolone antibacterial agents such as ciprofloxacin, and methods of making the same.

### BACKGROUND OF THE INVENTION

Ciprofloxacin (1-cyclopropyl-6-fluoro-1,4-dihydro-4-oxo-7-(1-piperazinyl)-3-quinolinecarboxylic acid) is a fluoroquinolone widely used in the treatment of bacterial infections (Rookaya Mather et al, American Journal of Ophthalmology, Vol. 133, No. 4, p463-466, 2002; P. C. Appelbaum et al, International Journal of Antimicrobial Agents, 16, 2000, p5-15). Fluoroquinolone antibacterial agents such as ciprofloxacin agents are preferred due to, among other reasons, their low MIC₉₀'s compared with conventional antibiotics and slower formation of resistant bacterial strains against them. For example, the MIC₉₀ of ciprofloxacin is generally around 0.5 μg/g whereas the MIC₉₀ of gentamicin is 10 μg/gm (Tai-Lee Ke et al, Journal of Ocular Pharmacology and Therapeutics, Vol. 17, No. 6, p555-562, 2001). Ciprofloxacin is a widely used in treat bacterial conjunctivitis of the eye and for treatment of corneal ulcers (Physicians Desk Reference; Steven J. Lichenstein, Contemporary Pediatrics, 2002, p16-19). The chemical structure of ciprofloxacin is: 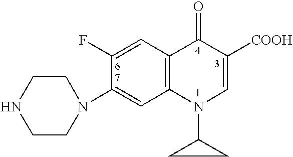
Ciprofloxacin is soluble in dilute (0.1N) hydrochloric acid and is practically insoluble in water and ethanol. The aqueous solubility of ciprofloxacin is 79 μg/mL (Danna L. Ross et al, International Journal of Pharmaceutics, 63 (1990), 237-250).

In order to achieve the 0.3% (3 mg/mL) concentration of ciprofloxacin necessary for therapeutic use in the currently marketed ciprofloxacin formulation of CILOXAN®, an acidic buffer is employed at pH 4.5. Upon administration of CILOXAN® ciprofloxacin formulation to the eye, frequent burning and stinging sensation has been clinically reported (Physicians Desk Reference). This is due to the acidic formulation pH of 4.5 and due to the invasive nature of the preservative, Benzalkonium chloride (BAK), used in the formulation of CILOXAN®. Also, the acidic pH of 4.5 leads to induced lachrymation, which in turn increases the drainage of the drug via the nasolachrymal duct (V. H. L. Lee et al, Journal of Ocular Pharmacology, 2 (1986), p67-108; Thorsteinn Loftsson et al, Advanced Drug Delivery Reviews, 36 (1999), p59-79; Marco Fabrizio Saettone, Pharmatech, 2002, p1-6). Such increased drainage is largely responsible for decreased availability of the therapeutic agent to the eye. This necessitates frequent and prolonged administration of the drug to eliminate the pathogens in question. It is therefore desirable to have a ciprofloxacin formulation which is formulated at a higher pH and which does not have the detrimental effects of the antimicrobial preservative currently being used.

Higher potency formulations of ciprofloxacin would be clinically desirable because they should increase the effective concentration of the drug that is locally delivered to the eye, which in turn will decrease the dosing regimen, increase patient compliance and decrease the duration of therapy (Steven J. Lichenstein, Contemporary Pediatrics, 2002, p16-19). Current techniques do not provide a feasible way to produce such higher potency formulations because further reductions in pH would lead to even more serious side effects. It is therefore desirable to have a ciprofloxacin drug formulation of higher potency (greater than 0.3%) and preferably formulated at a higher pH (higher than 4.5).

Formation of fluoroquinolone resistant strains of bacteria has been reported (Thomas J. Dougherty et al, DDT, Vol. 6, No. 10, 2001, p529-536). It is believed that this phenomenon is due to the decreased concentration of the therapeutic agent, concentrations below MIC₉₀ (minimum inhibitory concentration), in the presence of pathogens. “To avoid the development of resistance to topical antibiotics, high concentrations of a bactericidal drug with good solubility should be used at a dosing frequency that ensures that the drug concentrations are maintained above the MIC₉₀ of the suspected pathogens” (Steven J. Lichenstein, Contemporary Pediatrics, 2002, p16-19). It is therefore desirable to have formulations of higher potency (greater than 0.3%) that will maintain concentrations of the drug higher than MIC₉₀ in the eye. Such a formulation should increase therapeutic efficiency, decrease the likelihood of formation of resistant strains of bacteria, decrease the duration of therapy and decrease the dosing regimen.

Sulfoalkyl ether cyclodextrin derivatives and their use as solubilizing agents for water insoluble drugs for pharmaceutical administration has been disclosed by Stella et al. in U.S. Pat. No. 5,134,127 ('127 patent). Particular examples are sulfoalkylether cyclodextrins combined with various drugs, as ‘host-guest’ complexes. Exemplification has been achieved by the use of sulfoalkylether cyclodextrins in combination with digoxin, progesterone and testosterone. Among other things, this patent requires that the inclusion (clathrate complex) be formed prior to formulation. U.S. Pat. No. 5,376,645 ('645 patent) also by Stella et al is a continuation of the '127 patent. In addition to the exemplifications in the '127 patent, further examples in the '645 patent are phenyloin and naproxen.

U.S. Pat. No. 5,874,418 ('418 patent) and its continuation, U.S. Pat. No. 6,046,177, both by Stella et al., disclose the use of sulfoalkylether cyclodextrin based solid pharmaceutical formulations and their use. The composition comprises of a physical mixture of a sulfoalkylether cyclodextrin with a therapeutic agent, a major portion of which is not complexed to the cyclodextrin.

U.S. Pat. No. 5,324,718 and its continuation, U.S. Pat. No. 5,472,954, both by Thorsteinn Loftsson, provide a method for enhancing the complexation of a cyclodextrin with a lipophilic drug. The use of a water-soluble polymer such as HPMC as a co-solubilizer along with a cyclodextrin is disclosed. It one embodiment the polymer and the cyclodextrin are dissolved in the aqueous medium before the lipophilic drug is added and that solution be maintained from 30° C. to 150° C. for specified periods of time.

U.S. Pat. No. 5,855,916 to Chiesi describes the formation of soluble multicomponent inclusion complexes containing a base type drug, an acid and a cyclodextrin demonstrating enhanced water solubility. Exemplifications in the '916 patent include terfenadine, cinnarizine, domperidone, astemizole, ketoconazole, tamoxifene, clomifene and itraconazole as base type drugs.

PCT Application WO02/39993 describes a clear solution or gel preparation of a combination drug comprising an anti-inflammatory agent such as a corticosteroid, an anti-infective agent such as a fluoroquinolone a complexation enhancing polymer, and a solubilizer exhibiting an inclusion phenomenon.

### SUMMARY OF THE INVENTION

A first aspect of the present invention is an aqueous pharmaceutical composition comprising or consisting essentially of a fluoroquinolone active agent such as ciprofloxacin, cyclodextrin, a hydroxy acid, and water, the composition preferably having a pH between 5 and 7.

In some preferred embodiments, the composition further comprises or consists essentially of a soluble polymer.

In some embodiments, the composition further includes a steroidal or non-steroidal anti-inflammatory agent.

In some embodiments, the composition further comprises or consists essentially of another co-solubilizer such as a vitamin E derivative, detergents such as Tweens or pluronics, etc.

Compositions of the present invention are useful, among other things, for topical applications (e.g., to the eye, ear/ear passages, nose/nasal passages, etc.) and injectable applications (e.g., for subcutaneous, intramuscular, or intraperitoneal injection, etc.).

A second aspect of the present invention is a method of treating a bacterial infection and/or inflammation of an eye of a subject in need thereof, comprising topically administering a formulation as described above to the eye of the subject in an amount effective to treat the bacterial infection and/or inflammation.

A further aspect of the present invention is a method of treating a bacterial infection and/or inflammation of a topical surface of a subject such as an ear, nose or other skin surface need thereof, comprising topically administering a formulation as described above to the eye of the topical surface of subject in an amount effective to treat the bacterial infection and/or inflammation.

A further aspect of the present invention is an improved method of topically applying a pharmaceutical composition containing an active compound such as ciprofloxacin or other fluoroquinolone active agent to the eye of a subject in need thereof, which active compound precipitates from said composition on the eye, such as on the cornea, of the subject, the improvement comprising including a soluble polymer in said composition in an amount effective to reduce the precipitation of the active compound on the cornea of the subject.

A still further aspect of the present invention is an improved topical pharmaceutical composition containing an active compound (such as ciprofloxacin or other fluoroquinolone) used to topically apply said active compound to the eye of a subject in need thereof, which active compound precipitates from the composition on the eye or cornea of the subject, the improvement comprising including from 0.05 to 5% by weight of a soluble polymer in the composition to reduce the precipitation of the active compound on the eye or cornea of the subject.

The foregoing and other objects and aspects of the present invention are explained in detail in the drawings herein and the specification set forth below.

### BRIEF DESCRIPTION OF THE FIGURES

FIG. 1 shows that a combination formulation with all ingredients including hydroxypropylmethyl cellulose (HPMC), shows essentially no corneal precipitation in vitro.

FIG. 2 shows that a control combination formulation with all ingredients except HPMC leads to corneal precipitation in vitro.

FIG. 3 shows a generic formulation of Ciloxan exhibiting considerable reduction in assay values when exposed to radiation over a 24 h period. The figure further shows that when exposed to similar radiation over a 24 h period, the compositions that are part of these inventions are much more stable by comparison.

FIG. 4 shows the stability of a combination formulation when exposed to radiation over a 24 h period.

FIG. 5 shows the stability of a combination formulation when exposed to radiation over a 24 h period.

FIG. 6 shows that formulations that are part of these inventions are stable on long-term storage even under accelerated stability conditions. No precipitation of the active was observed throughout.

### DETAILED DESCRIPTION OF THE PREFERRED EMBODIMENTS

Subjects to be treated by the methods and compositions of the present invention are, in general, human subjects, but may also include other animal subjects, particularly mammalian subjects such as dogs, cats, horses and rabbits, for veterinary purposes.

As noted above, the present invention provides aqueous pharmaceutical compositions comprising:
(a) A fluoroquinolone such as Ciprofloxacin, typically included in an amount ranging from 1, 3, 5 or 8 mg/mL to 10, 20, 30, 50, 60 or 100 mg/mL of ciprofloxacin depending upon the intended use;
(b) optionally, but in some embodiments preferably, a steroid (including corticosteroids and prodrugs thereof) or a non-steroidal anti-inflammatory compound, which when included are present in an amount ranging from 1, 5 or 15 mg/mL up to 30, 60 or 100 mg/mL, depending upon the intended use;
(c) cyclodextrin (including combinations of cyclodextrins), typically included in an amount ranging from 1 to 7, 12, 15, 25, 30, 40 or 50% by weight;
(d) an acid, preferably a hydroxy acid, typically included in an amount ranging from 0.1 to 3, 10 or 25 molar equivalents thereof,
(e) optionally, but in some embodiments preferably, a water soluble polymer, which when included may be included in an amount ranging from about 0.05 to 1.5, 4 or 5 percent by weight of the aqueous formulation;
(f) optionally a co-solublizer such as a surfactant or Vitamin E TPGS, which when present is typically included in an amount of from 1, 2, or 5% up to 10 or 20% of the formulation; and
(h) water to balance; the formulation preferably having a pH between about 4, 4.5 or 5, up to about 7.

Lyophilized compositions which can be reconstituted with water to yield a composition as described above are also an aspect of the present invention.

Compositions in solid form comprised of ciprofloxacin, cyclodextrin and an acid as described above, and in the amounts as described above, are also an aspect of this invention.

The compositions are pharmaceutically acceptable in that they are sterile, pyrogen free, and suitable for topical or parenteral administration to a subject as described herein.

Fluoroquinolones that may be used to carry out the present invention include but are not limited to Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin etc. Such compounds are known and can be obtained from commercial sources or produced by techniques known in the art (See, e.g.,U.S. Pat. No. 4,670,444; Mather et al, American Journal of Ophthalmology, Vol. 133, No. 4, p463-466, 2002; P. C. Appelbaum et al, International Journal of Antimicrobial Agents, 16, 2000, p5-15; Tai-Lee Ke et al, Journal of Ocular Pharmacology and Therapeutics, Vol. 17, No. 6, p555-562, 2001; Physicans Desk Reference; Lichenstein, Contemporary Pediatrics, 2002, p16-19; Ross et al, International Journal of Pharmaceutics, 63 (1990), 237-250).

Ciprofloxacin (1-cyclopropyl-6-fluoro-1,4-dihydro-4-oxo-7-(1-piperazinyl)-3-quinolinecarboxylic acid) is known and can be obtained from commercial sources or produced by techniques known in the art (See, e.g., U.S. Pat. No. 4,670,444; Mather et al, American Journal of Ophthalmology, Vol. 133, No. 4, p463-466, 2002; P. C. Appelbaum et al, International Journal of Antimicrobial Agents, 16, 2000, p5-15; Tai-Lee Ke et al, Journal of Ocular Pharmacology and Therapeutics, Vol. 17, No. 6, p555-562, 2001; Physicians Desk Reference; Lichenstein, Contemporary Pediatrics, 2002, p16-19; Ross et al, International Journal of Pharmaceutics, 63 (1990), 237-250).

Steroid (or “steroidal”) compounds that can be used to carry out the present invention include but are not limited to cortisone, hydrocortisone, corticosterone, deoxycorticosterone, prednisolone, methylprednisolone, meprednisone, triamcinolone, paramethasone, fluprednisolone, betamethasone, dexamethazone, fludrocortisone, combinations thereof., etc., are known and can be obtained from commercial sources. The term “steroids” as used herein includes corticosteroids glucocorticoids, prodrugs of all thereof.

Non-steroidal antiinflammatory drugs that may be used to carry out the present invention include but are not limited to selected from aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone, acetaminophen, as well as COX-2 inhibitors such as nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, and S-2474, and mixtures thereof.

Any suitable cyclodextrin can be used to carry out the present invention, including α cyclodextrins, β cyclodextrins, γ cyclodextrins, and δ cyclodextrins (and which cyclodextrins may be in the form of derivatives such as sulfoalkylether cyclodextrins or hydroxyalkyl cyclodextrins). The amount of cyclodextrin will depend in part upon the amount of active ingredient to be included in the composition, but in general will be from about 1 to 7, 12, 30 or 40 percent by weight (for topical or injectable formulations) or from about 1 to 15, 25 or 50 percent by weight (for buccal/oral formulations).

Sulfoalkylether cyclodextrins that may be used to carry out the present invention may be of the following formula: 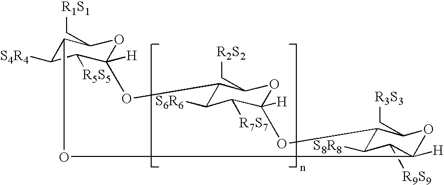
where:
n is 4, 5, 6 or 7 corresponding α, β, γ or δ cyclodextrin
R₁ through R₉ are independently —O— or a —O—(C₂ through C₆ alkylene)-SO₃ group, wherein at least one of R₁ and R₂ is independently a —O—(C₂ through C₆ alkylene)-SO₃ group, preferably a —O—(CH₂)ₘ—SO₃⁻ group, wherein m is 2 to 6 and S₁ through S₉ are independently pharmaceutically acceptable cations including H⁺, alkali metal cations, alkali earth metal cations and organic cations (WO 02/074200).

Hydroxyalkyl cyclodextrins used to carry out the present invention may be of the formula: 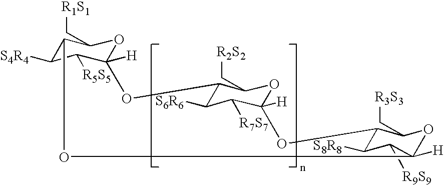
where:
n is 4, 5, 6 or 7 corresponding α, β, γ or δ cyclodextrin
R₁ through R₉ are independently —O— or a —O—(C₂ through C₆ alkylene)-O⁻-group, wherein at least one of R₁ and R₂ is independently a —O—(C₂ through C₆ alkylene) O⁻ group, preferably a —O—(CH₂)ₘ O⁻ group, wherein m is 2 to 6. The O— group can be attached to any of the methylene carbons. For eg: CH₂CH(O⁻)CH₃ and S₁ through S₉ are independently pharmaceutically acceptable cations including H⁺.

Any suitable hydroxy acid may be used to carry out the present invention, including but not limited to citric acid, ascorbic acid, malic acid, and tartaric acid, gluconic acid, lactic acid, treonic acid, and α, β, γ, δ or higher order aliphatic, alicyclic or aromatic hydroxy acids. The amount of hydroxy acid included will depend in part upon the amount of active ingredient to be included in the composition, but in general will be from about 0.1 up to about 3, 10 or 25 molar equivalents in the aqueous formulation.

While hydroxy acids are currently preferred, other acids, including mineral or organic acids such as phosphoric acid, sulfuric acid, hydrochloric acid, acetic acid, etc., may also be used.

Any suitable water soluble polymer may be used herein. In one preferred embodiment the polymer has an apparent viscosity of 1 to 100 mPa·s when dissolved in a 2% aqueous solution at 20° C. solution. Examples of suitable water soluble polymers include, but are not limited to, alkylcelluloses such as methylcellulose, hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectins such as sodium carboxymethylamylopectin; chitin derivatives such as chitosan; polysaccharides such as alginic acid, alkali metal and ammonium salts thereof, carrageenans, galactomannans, traganth, agar-agar, gum arabicum, guar gum and xanthan gum; polyacrylic acids and salts thereof, polymethacrylic acids and salts thereof, including methacrylate copolymers polyvinylpyrrolidone, copolymers of polyvinylpyrrolidone with vinyl acetate; polyalkylene oxides such as polyethylene oxide and polypropylene oxide and copolymers of ethylene oxide and propylene oxide; etc. Currently preferred is hydroxy propyl methyl cellulose, manufactured by Dow Chemical Industries, USA and also by Shin-Etsu Chemical Company, Japan.

Any suitable co-solubilizer may also be used to carry out the present invention. Such co-solubilizers include, but are not limited to, Pluronics (F-68, F-84 and P-103), Polaxamers, Vitamin E TPGS, Tweens (20, 60, 80), aliphatic alcohols and other agents known to those skilled in the art.

Compositions as described herein may include a tonicity modifier. Examples include, but are not limited to, NaCl, dextrose, glycerin, mannitol, and potassium chloride. In general, the tonicity of the composition is at least 100, 180 or 270 milli-Osmoles (mOsm), up to about 330, 540 to 600 mOsm, adjusted if desired by the inclusion of a tonicity modifier in the amount necessary to achieve an osmolarity within a range as given above. For example, where NaCl is utilized as a tonicity modifier, it may be included in an amount ranging from 0.01, 0.2 or 0.35 percent by weight, up to about 0.55, 3 or 10 percent by weight (with 0.45% by weight NaCl currently preferred).

Compositions as described herein may also contain a preservative. Any suitable preservative may be used to carry out the present invention, including but not limited to chlorobutanol, sorbic acid, salts of sorbic acid, EDTA, alcohol, bronopol, chlorhexidine, imidurea and sodium propionate. The amount of preservative is not critical, but will in general be from about 0.001 to about 0.5, 1 or 2% by weight of the aqueous formulation. Preservatives that are oppositely charged, such as BAK, are not suggested in formulations comprising of SBECD, also due to potential loss of activity due to complexation. Antimicrobial agents, such as parabens, which are capable of forming inclusion complexes them selves are also not preferred due to competitive displacement of the active.

As noted above, the ability of a molecule to be effectively solubilized by a cyclodextrin depends on variety of factors including the size of the cavity of the cyclodextrin being used, the size of the molecule etc., While some molecules effectively form a binary complex (drug-cyclodextrin complex), others might not. In a binary complex, addition of an appropriate amount of the guest molecule to an aqueous solution of the cyclodextrin at an appropriate concentration, at the appropriate temperature and agitation rate typically leads to the formation of a clear solution of the host-guest complex. In other words, the hydrophobic molecule will dissolve in an aqueous solution of the cyclodextrin without the help of a co-solubilizer such as ethanol (J. Pitha et al, International Journal of Pharmaceutics, 80, 1992, p253-258). Examples of such binary complexes are propofol-SBECD (WO 02/074200) and voriconazole-SBECD.

Methods of making. In one method of making formulations as described herein an aqueous solution of cyclodextrin is first prepared. To it is dispersed the drug, followed by addition of hydroxy acid. To it are added the water-soluble polymer, preservative, anti-oxidant or any other pharmaceutically acceptable additives. In another method, the polymer solution and the CD/drug/hydroxy acid solutions are separately prepared and mixed, followed by the addition of other pharmaceutically acceptable ingredients. Other methods include addition of any and all of the reagents in any combination or permutation possible. Another method includes mixing any or all the ingredients in the solid form before addition to water or any organic solvent. Various process parameters can be manipulated as desired, such as temperature control or modulation, agitation, sonication, autoclaving and pressurization or any other technique known to those skilled in the art. Another method includes preparing the liquid formulation as mentioned above or otherwise, and subsequently isolating the solid material by freeze drying, spray drying, spray-freeze drying, antisolvent precipitation, kneading, process involving supercritical fluid or near super critical fluid or any other methods for making a solid or liquid dosage form known to those skilled in the art.

Note that, in order for one to achieve the therapeutic concentration of 3 mg/mL, ciprofloxacin should be solubilized 37.5 fold. For higher potencies the solubilization has to be even higher (75 fold for a 6 mg/mL solution and 112.5 fold for a 9 mg/mL solution). Attempts at solubilization using 4.5% solution of SBECD led to an increase in ciprofloxacin solubility to 160 μg/mL. This corresponds to a two-fold increase in solubility and is far short of the 37.5 fold improvement that is desired. There are various reasons why the amount of CD in a solution formulation should be kept to a minimum (Thorsteinn Loftsson et al, Advanced Drug Delivery Reviews, 36 (1999), p59-79; Thorsteinn Loftsson et al, International Journal of Pharmaceutics, 225, 2001, p15-30). Achievement of the desired 3 mg/mL concentration is highly unlikely by simply increasing the percentage of SBECD (levels of pharmaceutical utility) in solution. In other words, formation of a binary complex of ciprofloxacin and SBECD to achieve the necessary solubilization is not feasible. Similar experiments with various concentrations of HPCD also proved that binary complex formation to achieve the desired solubility was not feasible.

However, in the presence of hydroxy acids, such as 0.2% of citric acid, ciprofloxacin complexed effectively with a 4% solution of SBECD. The solubilization achieved was over 112 fold in this particular case. Thus, one can effectively use a hydroxy acid, such as citric, ascorbic, malic, tartaric etc., as a co-solubilizer to achieve an increase in ciprofloxacin or other fluoroquinolone solubility. Such an increase is in a preferred embodiment is synergistic and cannot be achieved by simple binary complexation. Such multicomponent complexes involving ciprofloxacin/SBECD/citric acid have not been reported in literature. One should note that solubility of ciprofloxacin in 0.2% citric acid solution alone is far less (<3 mg) than what is achieved by synergistic multicomponent complexation. Similar synergistic complexation is also clearly evident in formulations comprising of Gatifloxacin. For higher potency formulations of Gatifloxacin (0.6%, 0.9% or higher) synergistic multicomponent formulations are extremely critical since it allows the achievement of higher concentrations without reduction in pH compared to the commercial formulation (Zymar, 0.3% Gatifloxacin, pH=ca. 6).

The amount of citric acid required to effect the required solubilization is also an aspect of this invention. If one has to use to enough citric acid such that the pH of the invention is same or less than the commercial formulation of 4.5, the utility of the invention will be somewhat reduced. Equivalence determination studies demonstrated that this was not the case. For effective solubilization one needs to use only a minimum of 0.5 mole equivalents of citric acid for every mole equivalents of ciprofloxacin or other fluoroquinolone. Accordingly in this invention, the pH of a 6 mg/mL formulation (doubly potent) is at about pH 5.0. This is about 0.5 units higher than the commercial formulation which is only half potent. The clinical benefits of this invention are readily apparent.

Reduction of corneal precipitation. Corneal precipitation has been reported as an undesirable side effect to patients using CILOXAN® ciprofloxacin formulations for conjunctivitis and especially for corneal ulcers (H. M. Liebowitz, American Journal of Ophthalmology, 1991, 112, 34S-47; D. J. Parks et al, American Journal of Ophthalmology, 1993, 115, 471-477; R. A. Eiferman, Journal of Cataract and Refractive Surgery, 2001, 27, p1701-1702; H. N. Madhavan, Cornea, 1999, 18:549-552). As has been alluded to before, this phenomenon occurs when the pH of the eye is higher than the pKa of ciprofloxacin (pKa=6.09, typically around 6 minutes for a true solution formulation) and there is sufficient concentration of the drug still left in the eye. Avoidance of such precipitates is all the more important in these inventions since these inventions are at least as potent as the commercial formulation (3 mg/mL) and preferred formulations are doubly or triply potent or greater compared to the commercial formulation. Corneal precipitations can be observed in vitro using the in vitro tear turn-over model. The fact that the commercial CILOXAN® ciprofloxacin formulation does indeed lead to corneal precipitation has been independently demonstrated by Allergan Inc (B. A. Firestone et al, International Journal of Pharmaceutics, 164 (1998), p119-128). These data are confirmed herein.

Thus, in yet another embodiment of this invention, the use of a water soluble polymer such as described above to reduce, minimize, control prevent corneal precipitation of the drug at pH's higher than the pKa of the drug. The preferred polymers are MC, CMC, HPMC, PVP, PVA and poloxamers. The most preferred polymers are HPMC and PVA.

In another embodiment of this invention, aqueous based combination formulations, of fluoroquinolones and anti-inflammatory agents, such as steroids, corticosteroids or non-steroidal agents are included. Such formulations are not reported in literature or available commercially. Due to the sparse water solubility of fluoroquinolones and steroids, manufacture of an aqueous solution of these drugs is not feasible. This invention provides for a way of manufacturing a true aqueous based solution of these two drugs. The formulations shall be of higher potency and with pH's between 5 and 7.

Compositions of the present invention can be used to treat subjects as described herein in a manner analogous to that utilized with present fluoroquinolone compositions. Topical compositions may be administered to the eye of a subject as droplets as desired to treat eye infections. Oral or injectable formulations may be likewise administered in accordance with known techniques.

Bacterial infections of the eye and/or inflammations which may be treated by the topical or ophthalmic methods and compositions described herein include but are not limited to infections with gram-Positive bacteria such as Staphylococcus aureus (including methicillin-susceptible and methicillin-resistant strains), Staphylococcus epidermidis, Streptococcus pneumoniae, Streptococcus (Viridans Group), as well as infections with gram-negative bacteria such as Haemophilus influenzae, Pseudomonas aeruginosa, and Serratia marcescens.

Other bacterial infections, including but not limited to bacterial infections of the skin, joints, and airways, which may be treated with the intravenous methods and compositions described herein include infections with aerobic gram-positive microorganisms such as Enterococcus faecalis, Staphylococcus aureus (methicillin-susceptible strains only), Staphylococcus epidermidis (methicillin-susceptible strains only), Staphylococcus saprophyticus, Streptococcus pneumoniae (penicillin-susceptible strains), and Streptococcus pyogenes, and infections with Aerobic gram-negative microorganisms such as Citrobacter diversus Morgan ella morgan ii, Citrobacter freundii Proteus mirabilis, Enterobacter cloacae Proteus vulgaris, Escherichia coli Providencia rettgeri, Haemophilus influenzae Providencia stuartii, Haemophilus parainfluenzae Pseudomonas aeruginosa, Klebsiella pneumoniae Serratia marcescens, Moraxella catarrhalis, Burkolderia picketti, and inhalation anthrax.

The present invention is explained in greater detail in the Examples below, where “CD” means cyclodextrin; “SBE7-β-CD” means sulfobutylether7-β-cyclodextrin; “HPCD” means 2-hydroxypropylether-α-cyclodextrin; “HPMC” means hydroxypropylmethyl cellulose; “PVA” means polyvinyl alcohol.

### EXAMPLE 1

### Formulation of Ciprofloxacin and Sulfoalkylether Cyclodextrin

The following formulation was made according to the following procedure. SBE7-β-CD was dissolved in distilled, deionized water to obtain a concentration of about 2%. While the aqueous CD solution was being stirred, ciprofloxacin, in amounts that would eventually provide a 3 mg/mL solution, was dispersed into it. This was followed by the addition of citric acid (0.1 eq to 10.0 eq in relation to molar concentration of ciprofloxacin). The solution was stirred until it became clear. No viscosity enhancing agents, preservatives or other pharmaceutically acceptable ingredients were added. The solution was brought up to volume or weight with distilled water under agitation. Results: pH=5.2; Osmolality=150 mOsm.

### EXAMPLE 2

### Formulation of Ciprofloxacin and Sulfoalkylether Cyclodextrin

Appropriate amounts of SBE7-β-CD was dissolved in distilled, deionized water to obtain a concentration of about 1% to about 30%. While the aqueous CD solution was being stirred, ciprofloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. No viscosity enhancing agents, preservatives or other pharmaceutically acceptable ingredients were added. The solution was brought up to volume or weight with distilled water under agitation.

### EXAMPLE 3

### Formulation of Ciprofloxacin and Sulfoalkylether Cyclodextrin with Polymer and Preservative

Appropriate amounts of SBE7-O-CD was dissolved in distilled, deionized water to obtain a concentration of about 1% to about 30%. While the aqueous CD solution was being stirred, ciprofloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. Water soluble polymer, hydroxypropylmethyl cellulose (E50), was added to the solution such that the concentration of the polymer is about 0.1% to 10%. Preservative, chlorobutanol, was added such that its concentration is between 0.1% to 1%. Tonicity modifiers such as sodium chloride are added, if needed. The solution was brought up to volume or weight with distilled water under agitation.

### EXAMPLE 4

### Formulation of Ciprofloxacin and Sulfoalkylether Cyclodextrin with Polymer and Preservative

Appropriate amounts of SBE7-β-CD was dissolved in distilled, deionized water to obtain a concentration of about 1% to about 30%. While the aqueous CD solution was being stirred, ciprofloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. Water soluble polymer, hydroxypropylmethyl cellulose (E50), was added to the solution such that the concentration of the polymer is about 0.1% to 10%. Preservative, chlorobutanol, was added such that its concentration is between 0.1% to 1%. The solution was brought up to volume or weight with distilled water under agitation. Tonicity modifiers such as sodium chloride are added, if needed. This solution was filtered through a filter of 0.45 μm or lower porosity and freeze-dried.

### EXAMPLE 5

Appropriate amounts of SBE7-p-CD was dissolved in distilled, deionized water to obtain a concentration of about 1% to about 30%. While the aqueous CD solution was being stirred, ciprofloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. A water soluble polymer, hydroxypropylmethyl cellulose (viscosities ranging from 2 cps to 40,000 cps), was added to the solution such that the concentration of the polymer is about 0.1% to 10%. A preservative, chlorobutanol, is added such that its concentration is between 0.1% to 1%. Tonicity modifiers such as sodium chloride are added, if needed. The solution is brought up to volume or weight with distilled water under agitation. This solution is filtered through a filter of 0.45 μm or lower porosity and further processed to obtain a liquid or solid formulation.

### EXAMPLE 6

### Ciprofloxacin HPLC Method

This example describes methods for the analysis of ciprofloxacin content in compositions of the invention by high performance liquid chromatography (HPLC).
Column: Agilent Zorbax Eclipse XDB-C 18, 4.6×150 mm, 3.5μ
Mobile Phase: 87:13, 0.025 M phosphoric acid pH 3.0: Acetonitrile
Injection volume: 10 μL
UV detection @ 278 nm
Flow rate: 1 mL/min
Column temperature: 40° C.
   Precision:
   Response of a 0.05 mg/mL solution
   % RSD (n=10)=0.6%
Accuracy:
   Compared to a second standard solution of same concentration=98.6%
      Linearity:

Performed 10 injections of Ciprofloxacin standard 0.05 mg/mL at 5.0, 7.5, 10, 12.5 and 15.0 μL volumes, which corresponded to 0.25, 0.375, 0.5, 0.625, and 0.75 μg of Ciprofloxacin loaded onto the column. These values corresponded to 50 to 150% of the nominal concentration of 0.05 mg/mL. The % RSD₁₀ of each set of injections were all <1.3%.

LOD/LOQ:

Performed 6 injections of solutions of ciprofloxacin ranging in concentrations from 0.0001 to 0.01 mg/mL in the attempt to get an estimation of LOD/LOQ.
LOD=10 (SD/S)
10 μl of 0.0001 mg/mL (0.001 μg column load) X₆=18373, SD=1059
LOQ=10 (1059/7.0×10⁶)=0.0015 μg
LOD=3.3 (1059/7.0×10⁶)=0.0005 μg

### EXAMPLE 7

### Degradation Study

The purpose of this example was to demonstrate that the HPLC assay described above can also be utilized as stability indicating assay method.

Procedure: Purposely cause degradation of a solution of Ciprofloxacin by exposing a solution of the active to 2 M methanolic acid (HCl in methanol) and 0.2 M NaOH in water under ambient and heated conditions. Combine 1.0 mg/mL of Ciprofloxacin with 2 M methanolic acid and also combine 1.0 mg/mL of Ciprofloxacin with 0.2 M NaOH in water to get a final concentration of 0.5 mg/mL Ciprofloxacin. Store solutions at ambient conditions and at 80° C. for approximately 24 hours. Control samples were also prepared by diluting 1.0 mg/mL of Ciprofloxacin with the appropriate amount of solvent (either methanol or water) and will also be stored under ambient and heated conditions.

Method: Same as assay method.

Results: Under the conditions of heating with or without base or acid, new peaks were detected at the following relative retention times (RRT's, relative to the main ciprofloxacin peak). 0.27, 0.36, 0.55, 0.60, 0.68 and 0.72.

Conclusion: The results of the degradation studies show the presence of additional, well-resolved peaks indicating that this method can be utilized for both assay and stability indicating purposes.

### EXAMPLE 8

### Formulation of Gatifloxacin and Sulfoalkylether Cyclodextrin

The following formulation was made according to the following procedure. SBE7-β-CD was dissolved in distilled, deionized water to obtain a concentration of about 3%. While the aqueous CD solution was being stirred, Gatifloxacin, in amounts that would eventually provide a 6 mg/mL solution, was dispersed into it. This was followed by the addition of citric acid (0.1 eq to 10.0 eq in relation to molar concentration of Gatifloxacin). The solution was stirred until it became clear. No viscosity enhancing agents, preservatives or other pharmaceutically acceptable ingredients were added. The solution was brought up to volume or weight with distilled water under agitation. Results: pH=ca. 6; Osmolality=ca. 150 mOsm.

### EXAMPLE 9

### Formulation of Gatifloxacin and Sulfoalkylether Cyclodextrin

Appropriate amounts of SBE7-β-CD was dissolved in distilled, deionized water to obtain a concentration of about 1% to about 30%. While the aqueous CD solution was being stirred, Gatifloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. No viscosity enhancing agents, preservatives or other pharmaceutically acceptable ingredients were added. The solution was brought up to volume or weight with distilled water under agitation.

### EXAMPLE 10

### Formulation of Gatifloxacin and Sulfoalkylether Cyclodextrin with Polymer and Preservative

Appropriate amounts of SBE7-β-CD was dissolved in distilled, deionized water to obtain a concentration of about 1% to about 30%. While the aqueous CD solution was being stirred, Gatifloxacin, in amounts that would eventually provide a concentration between 1 mg/mL and 60 mg/mL, was dispersed into it. This was followed by the addition of citric acid (0.1 eq to 10.0 eq in relation to molar concentration of Gatifloxacin). The solution was stirred until it became clear. Water soluble polymer, hydroxypropylmethyl cellulose, was added to the solution such that the concentration of the polymer is about 0.1% to 10%. Preservative, chlorobutanol, was added such that its concentration is between 0.1% to 1%. Tonicity modifiers such as sodium chloride are added, if needed. The solution was brought up to volume or weight with distilled water under agitation.

### EXAMPLE 11

### Formulation of Gatifloxacin, Hydrocortisone, Hydroxypropyl Cyclodextrin and Sulfoalkylether Cyclodextrin with Polymer and Preservative

Appropriate amounts of SBE7-β-CD and HPCD was dissolved in distilled, deionized water to obtain a concentration of about 1% to about 30%. While the aqueous CD solution was being stirred, Gatifloxacin, in amounts that would eventually provide a concentration between 1 mg/mL and 60 mg/mL, was dispersed into it. This was followed by the addition of citric acid (0.1 eq to 10.0 eq in relation to molar concentration of ciprofloxacin). The solution was stirred until it became clear. This followed by the dispersion of hydrocortisone, in amounts that would eventually provide a concentration between 1 mg/mL and 60 mg/mL. After the solution clarifies, water soluble polymer, hydroxypropylmethyl cellulose, was added to the solution such that the concentration of the polymer is about 0.1% to 10%. Preservative, chlorobutanol, was added such that its concentration is between 0.1% to 1%. The solution was brought up to volume or weight with distilled water under agitation. Tonicity modifiers such as sodium chloride are added, if needed. This solution was filtered through a filter of 0.45 μm or lower porosity and further processed to obtain a solid or liquid formulation.

### EXAMPLE 12

Appropriate amounts of HPCD and SBE7-β-CD was dissolved in distilled, deionized water to obtain a concentration of about 1% to about 30%. While the aqueous CD solution was being stirred, Gatifloxacin, in amounts that would eventually provide a concentration between 1 mg/mL and 60 mg/mL, was dispersed into it. This was followed by the addition of citric acid (0.1 eq to 10.0 eq in relation to molar concentration of Gatifloxacin). The solution was stirred until it became clear. After dispersion of hydrocortisone and clarification of the solution, water soluble polymer, hydroxypropylmethyl cellulose (viscosities ranging from 2 cps to 40,000 cps), was added to the solution such that the concentration of the polymer is about 0.1% to 10%. Preservative, chlorobutanol, was added such that its concentration is between 0.1% to 1%. Tonicity modifiers such as sodium chloride are added, if needed. The solution was brought up to volume or weight with distilled water under agitation. This solution was filtered through a filter of 0.45 μm or lower porosity and further processed to obtain a liquid or solid formulation.

### EXAMPLE 13

### Gatifloxacin HPLC Method

Column: Agilent Zorbax Eclipse XDB-C18, 4.6×150 mm, 3.5μ
Mobile Phase: 85:15, 0.025 M phosphoric acid pH 3.0 (with TEA): Acetonitrile
Injection volume: 10 μL
UV detection @ 293 nm
Flow rate: 1 mL/min
Column Temperature: 40° C.
   Precision

A 0.1005 mg/mL Standard Solution had a response of 0.34 AU.

There was 0.1% RSD between ten injections.

Accuracy

Compared to a second standard solution, the control fell between 99.8-100.2% over 7 (0, 1, 5, 7) days.

### Linearity

Five Gatifloxacin standard solutions were prepared of concentration 0.050025, 0.07575, 0.1005, 0.1255, and 0.1515 mg/mL and injected ten times each. These values corresponded to 50 to 150% of the nominal concentration of 0.1 mg/mL. The % RSD₁₀ of each set of injections were all <0.2%.

### LOD/LOQ

Performed 6 injections of Ciprofloxacin ranging from 0.00002367 to 0.0007575 mg/mL in the attempt to get an estimation of LOD/LOQ.

LOD=10 (SD/S) LOQ=3.3(SD/S)

Used the 0.00002367 mg/mL (0.0002367 μg column load) X₆=2406, SD=275
LOQ=10(275/5.0×10⁶)=0.00055 μg
LOD=3.3(275/5.0×10⁶)=0.0001815 μg

### EXAMPLE 14

### Soluble Polymers and Corneal Precipitation

The prevention of corneal precipitation with a soluble polymer such as HPMC is a further object of the present invention. At pH's higher than the pKa of fluoroquinolones, HPMC, and to a very slightly reduced degree PVA, is able to prevent corneal precipitation even at high concentrations, in vitro. These concentrations are far higher than the solubility of fluoroquinolones in solutions of HPMC and PVA at concentrations in the formulations. This result is particularly unexpected since various published reports have stated that in order for a water soluble polymer to co-solubilize a hydrophobic drug in the presence of a cyclodextrin, micelle formation is necessary (A. M. Sigurdardottir et al, International Journal of Pharmaceutics, 126, 1995, p73-78; J. K. Kristinsson et al, Investigations in Ophthalmology Visual Sciences, 37, 1996, p1199-1203; Thorsteinn Loftsson et al, Advanced Drug Delivery Reviews, 36 (1999), p59-79). The reports further state that such micelle formation of the cyclodextrin and polymer is possible only by aggressive processing such as autoclaving at 120° C. or sonication at 80° C. for hours. Our process involves nothing but benign agitation.

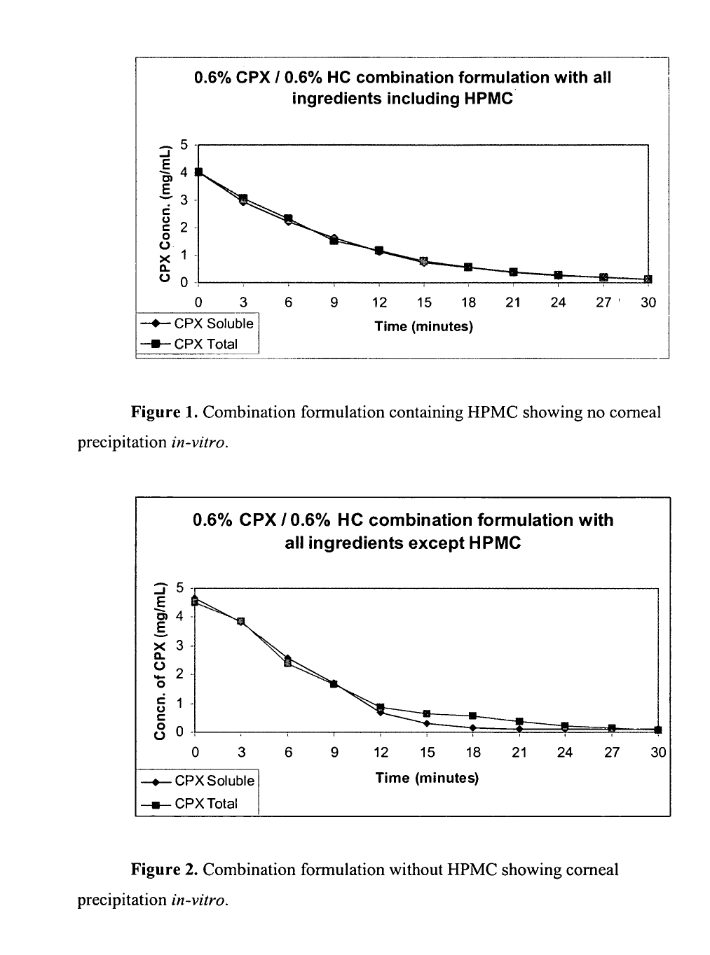

FIG. 1 shows that a combination formulation (Ciprofloxacin/Hydrocortisone, 0.6%/0.6%) with all ingredients including HPMC, shows essentially no corneal precipitation in vitro. This is inferred from the fact that the total and soluble concentrations are same, as the pH of the tear film gets adjusted to normal lachrymal pH as a function of time, within experimental error. While this in vitro tear turn-over study simulated first-order nasolachrymal drainage and equilibration to lachrymal pH as a function of time, it does not simulate other important parameters such as increase in residence time in the eye as the viscosity of the formulation is increased and induced lachrymation as a function of the formulation.

FIG. 2 shows that a control combination formulation (Ciprofloxacin/Hydrocortisone, 0.6%/0.6%) with all ingredients except HPMC leads to corneal precipitation in vitro. This is inferred from the fact that at pH's higher than 6.1, the total concentration is much higher than the soluble concentration (as high as 200%). It is visually obvious that precipitation is commenced at pH's higher than the pKa, in formulations without the “pH independent precipitation inhibitor”. The solution becomes very cloudy and the drug can be visually observed as fine particles suspended in solution. The experimental logic and design has already been published for these in vitro tear turn-over experiments (B. A. Firestone et al, International Journal of Pharmaceutics, 164 (1998), p119-128).

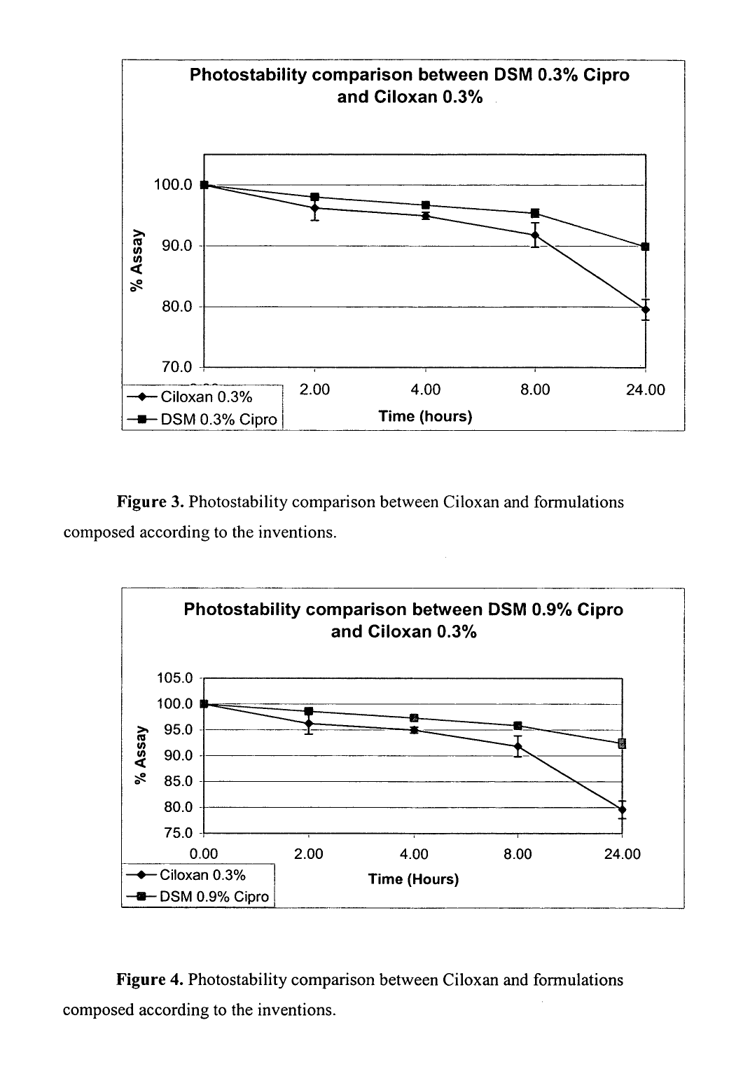

FIG. 3 shows that Ciloxan (0.3% Ciprofloxacin Hydrochloride) when exposed to a photostability chamber (ICH conditions), undergoes substantial degradation over a 24 hr period. The figure also shows that 0.3% ciprofloxacin formulation composed according to the inventions given above are considerably more stable than Ciloxan itself.

FIG. 4 shows that Ciloxan (0.3% Ciprofloxacin Hydrochloride) when exposed to a photostability chamber (ICH conditions), undergoes substantial degradation over a 24 hr period. The figure also shows that a triply potent ciprofloxacin formulation composed according to the inventions is considerably more stable than than Ciloxan itself.

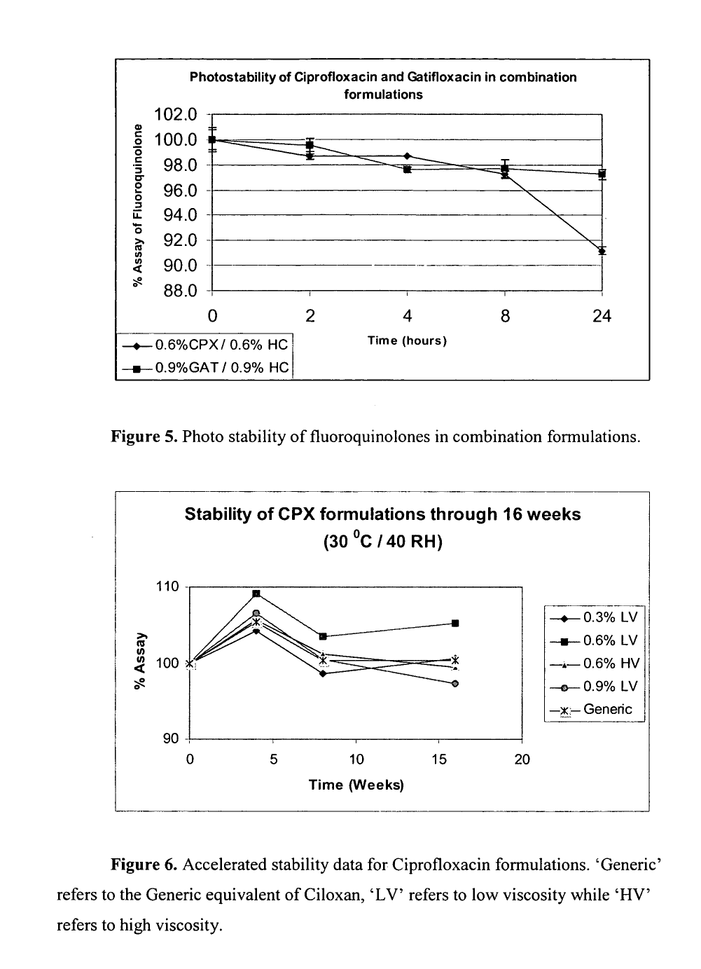

FIG. 5 shows the photo stability of ciprofloxacin and gatifloxacin in ciprofloxacin/hydrocortisone and gatifloxacin/hydrocortisone combination formulations respectively.

FIG. 6 shows that formulations that are part of these inventions are stable on long-term storage even under accelerated stability conditions. No precipitation of the active was observed throughout.

It is fairly well documented than fluoroquinolone solutions such as ciprofloxacin solutions are stable at acidic pH's (<5) and that considerable degradation occurs at higher pH's (K Torniainen et al, International Journal of Pharmaceutics, 132, 1996, p53-61; K Torniainen et al, Journal of Pharmaceutical and Biomedical Analysis, 16, 1997, p439-445; K Torniainen et al, Journal of Pharmaceutical and Biomedical Analysis, 15, 1997, p887-894). Whilst no buffering agents have been added to increase stability or adjust buffering capacity, our invention showed unexpected buffering. The buffer capacity for a formulations are generally in the range of 0.001 or higher. It is believed that, in addition to the formation of an inclusion complex, the formulations mentioned in these inventions are further stabilized by this coincidental buffering.

The foregoing is illustrative of the present invention, and is not to be construed as limiting thereof. The invention is described by the following claims, with equivalents of the claims to be included therein.

1. An aqueous pharmaceutical composition comprising:
from 5 to 100 mg/mL of a fluoroquinolone active agent;
from 0 to to 100 mg/mL of a steroidal or non-steroidal anti-inflammatory agent;
from 1 to 40% by weight of cyclodextrin;
from 0.1 to 25 molar equivalents of a hydroxy acid; and
water to balance,
said formulation having a pH between 4.5 and 7.

2. The composition according to claim 1, wherein said cyclodextrin is selected from the group consisting of α cyclodextrins, β cyclodextrins, γ cyclodextrins, and δ cyclodextrins.

3. The composition according to claim 1, wherein said cyclodextrin is selected from the group consisting of sulfoalkylether cyclodextrins and hydroxyalkyl cyclodextrins.

4. The composition according to claim 1, wherein said hydroxy acid is selected from the group consisting of citric acid, ascorbic acid, malic acid, and tartaric acid.

5. The composition according to claim 1, further comprising from 0.001 to 2 percent by weight of a preservative.

6. The composition according to claim 1, further comprising a preservative selected from the group consisting of chlorobutanol, sorbic acid, and EDTA.

7. The composition according to claim 1, further comprising: from 0.05 to 5% by weight of a soluble polymer.

8. The composition according to claim 7, wherein said soluble polymer is selected from the group consisting of methylcellulose, carboxymethylcellulose, hydroxypropylmethylcellulose, polyvinylpyrrolidone, polyvinyl alcohol, and poloxamers.

9. The composition according to clainm 1, wherein said fluoroquinolone is selected from the group consisting of Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin and combinations thereof.

10. The composition according to claim 1, wherein said steroidal or non-steroidal anti-inflammatory compound is a steroidal compound and is selected from the group consisting of cortisone, hydrocortisone, corticosterone, deoxycorticosterone, prednisolone, methylprednisolone, meprednisone, triamcinolone, paramethasone, fluprednisolone, betamethasone, dexamethazone, fludrocortisone, and combinations thereof.

11. The composition according to claim 1, wherein said steroidal or non-steroidal anti-inflammatory compound is a non-steroidal compound and is selected from the group consisting of aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone, acetaminophen, nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, and S-2474, and combinations thereof.

12. A method of treating a bacterial infection of an eye of a subject in need thereof, comprising topically administering a formulation according to claim 1 to the eye of said subject in an amount effective to treat said bacterial infection.

13. A pharmaceutical formulation comprising:
from 5 to 100 mg/mL of a fluoroquinolone active agent;
from 0 to to 100 mg/mL of a steroidal or non-steroidal anti-inflammatory agent;
from 1 to 40% by weight of cyclodextrin;
from 0.1 to 25 molar equivalents of a hydroxy acid.

14. A pharmaceutical formulation according to claim 13 in lyophilized form which when reconstituted with water produces an aqueous pharmaceutical composition having a pH between 4.5 and 7 and comprising:
from 5 to 100 mg/mL of a fluoroquinolone active agent;
from 1 to 40% by weight of cyclodextrin;
from 0.1 to 25 molar equivalents of a hydroxy acid; and
water to balance.

15. The composition according to claim 13, wherein said cyclodextrin is selected from the group consisting of α cyclodextrins, β cyclodextrins, γ cyclodextrins, and δ cyclodextrins.

16. The composition according to claim 13, wherein said cyclodextrin is selected from the group consisting of sulfoalkylether cyclodextrins and hydroxyalkyl cyclodextrins.

17. The composition according to claim 13, wherein said hydroxy acid is selected from the group consisting of citric acid, ascorbic acid, malic acid, and tartaric acid.

18. The composition according to claim 13, further comprising from 0.001 to 2 percent by weight of a preservative.

19. The composition according to claim 18, said preservative selected from the group consisting of chlorobutanol, sorbic acid, and EDTA.

20. The composition according to claim 14, further comprising: from 0.05 to 5% by weight of a soluble polymer.

21. The composition according to claim 21, wherein said soluble polymer is selected from the group consisting of methylcellulose, carboxymethylcellulose, hydroxypropylmethylcellulose, polyvinylpyrrolidone, polyvinyl alcohol, and poloxamers.

22. The composition according to clainm 13, wherein said fluoroquinolone is selected from the group consisting of Gatifloxacin, Moxifloxacin, Sitafloxacin, Lomefloxacin, Grepafloxacin, Gemifloxacin, Norfloxacin, Ofloxacin, Levofloxacin, Trovafloxacin, Ciprofloxacin and combinations thereof.

23. The composition according to claim 13, wherein said steroidal or non-steroidal anti-inflammatory compound is a steroidal compound and is selected from the group consisting of cortisone, hydrocortisone, corticosterone, deoxycorticosterone, prednisolone, methylprednisolone, meprednisone, triamcinolone, paramethasone, fluprednisolone, betamethasone, dexamethazone, fludrocortisone, and combinations thereof.

24. The composition according to claim 13, wherein said steroidal or non-steroidal anti-inflammatory compound is a non-steroidal compound and is selected from the group consisting of aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone, acetaminophen, nimesulide, NS-398, flosulid, L-745337, celecoxib, rofecoxib, SC-57666, DuP-697, parecoxib sodium, JTE-522, valdecoxib, SC-58125, etoricoxib, RS-57067, L-748780, L-761066, APHS, etodolac, meloxicam, and S-2474, and combinations thereof. thereof.

25. In a method of topically applying a pharmaceutical composition containing an active compound to the eye of a subject in need thereof, which active compound precipitates from said composition on the cornea of said subject, the improvement comprising: including a soluble polymer in said composition in an amount effective to reduce the precipitation of said active compound on the cornea of said subject.

26. The method according to claim 25, wherein said soluble polymer is selected from the group consisting of methylcellulose, carboxymethylcellulose, hydroxypropylmethylcellulose, polyvinylpyrrolidone, and polyvinyl alcohol, and poloxamers.

27. The method according to claim 25, wherein said active compound is a fluoroquinolone.

28. The method according to claim 25, said pharmaceutical composition further comprising a steroidal or non-steroidal anti-inflammatory compound.

29. In a topical pharmaceutical composition containing an active compound used to topically apply said active compound to the eye of a subject in need thereof, which active compound precipitates from said composition on the cornea of said subject, the improvement comprising: including from 0.05 to 5% by weight of a soluble polymer in said composition in an amount effective to reduce the precipitation of said active compound on the cornea of said subject.

30. The composition according to claim 29, wherein said soluble polymer is selected from the group consisting of methylcellulose, carboxymethylcellulose, hydroxypropylmethylcellulose, polyvinylpyrrolidone, and polyvinyl alcohol, and poloxamers.

31. The composition according to claim 29, wherein said active compound is a fluoroquinolone.

32. The composition according to claim 29, said composition further comprising a steroidal or non-steroidal anti-inflammatory compound.
